(19) United States
(12) Patent Application Publication
(10) Pub. No.: US 2015/0157787 A1
(43) Pub. Date: Jun. 11, 2015

(54) NEEDLE GUIDE AND RELATED SYSTEMS AND METHODS

(71) Applicant: W. L. Gore & Associates, Inc., Newark, DE (US)

(72) Inventors: Edward H. Cully, Flagstaff, AZ (US); Jeffrey B. Duncan, Flagstaff, AZ (US); James L. Goepfrich, Flagstaff, AZ (US)

(21) Appl. No.: 14/558,153

(22) Filed: Dec. 2, 2014

Related U.S. Application Data
(60) Provisional application No. 61/912,454, filed on Dec. 5, 2013.

(51) Int. Cl. A61M 5/158

(52) U.S. Cl. CPC A61M 5/158

(57) ABSTRACT

Illustrative embodiments of the present disclosure comprise devices, systems and methods for improved dialysis cannulation. Embodiments of the present disclosure can provide for a consistent cannulation entry site, entry angle and/or depth. Embodiments of the present disclosure can also be configured to maintain a drug at the cannulation entry site and/or maintain pressure at the cannulation entry site.

### TECHNICAL FIELD

The present disclosure relates to devices, systems and methods for improved dialysis cannulation.

### BACKGROUND

Maintenance hemodialysis for end stage renal disease (ESRD) patients requires a reliable means of repetitive access to large blood vessels that are capable of rendering rapid extracorporeal blood flow to an artificial kidney. Typically, an artery and vein are sutured together to form a fistulae which enlarges to a point of maturity over several months. Synthetic grafts may also used.

Hemodialysis patients receiving dialysis treatment via native vein arteriovenous fistulae and synthetic grafts typically undergo puncture of skin, subcutaneous tissue, and vascular access with 14-17 gauge needles two to three times weekly. When the procedure is finished and the needles are removed from the skin, many patients bleed from the puncture site for an extended period of time such that the standard treatment involves post-hemodialysis compression at the site for at least 15-20 minutes.

Problems are commonly associated with repeated vascular access, i.e., access to circulation, and include hyperplasia, thrombosis, hematoma, venous stenosis, arterial stenosis, vascular occlusion, infection, and morbidity. In those situations where anatomic lesions can be identified, the pathology has been found to be intimal hyperplasia. Other causes of vascular access complications include: venous or arterial stenosis and infection. These complications with vascular access sites lead to blocking or narrowing of vascular access sites, which in turn result in an increased incidence of surgery to repair, replace, or create new vascular access sites. Once a vascular access site fails, a new site (new location on the patient) is used to regain access to the circulation. The human body has a limited amount of these viable sites.

Hemodialysis vascular access is also a major risk factor for infection and bacteremia, caused mostly by staphylococcal organisms. These infections and bacteremia lead to complications such as degradation in vascular access sites and surgical replacement of vascular access sites. Other complications can include infectious endocarditis, septic arthritis, epidural abscess, septic pulmonary emboli, and osteomyelitis.

Lack of skilled dialysis technicians and self-cannulation (home hemodialysis) also contribute to excessive damage caused to both native fistulae and grafts by misplaced cannulations.

Preserving access function and long-term vascular access is essential for the care of dialysis patients, particularly now that high-efficiency dialysis places even more demands on access function, and with increasing numbers of older, sicker patients entering the ESRD program with limited access sites. Vascular access complications remain the single greatest cause of morbidity and account for approximately one third of all admissions and hospitalization days in the hemodialysis population.

Therefore, systems, methods and devices are still needed which will reduce vascular access complications such as mentioned above. The reduction of complications of existing sites in turn preserves the remaining access sites of the patient for use at a later date. The present disclosure addresses this need.

### SUMMARY

Particular embodiments of the subject matter described in this specification can be implemented so as to realize one or more of the following advantages. A consistent dialysis cannulation entry site, entry angle and/or depth can be provided. A drug can be maintained at the entry site. Pressure can be maintained at the entry site.

In one general aspect, a needle guide is provided that comprises a base configured to provide an interface for the needle guide to couple to a surface, a cavity defining a void above the surface to which the base is coupled, a needle path defining a bore through which a needle can pass to a consistent entry site at a consistent entry angle, and a needle stop.

In various implementations, the void comprises a filler, for example, a pain reliever, anti-bacterial agent, anticoagulant and/or a combination of the foregoing. In various implementations, the filler exerts pressure at the entry site when a needle is removed therefrom, and thereby prevents excessive bleeding and reduces the need for prolonged digital pressure

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A illustrates a needle guide in accordance with illustrative embodiments of the present disclosure;

FIG. 1B is a cross-section of what is illustrated in FIG. 1A;

FIG. 2A illustrates a needle guide comprising a plurality of needle paths in accordance with illustrative embodiments of the present disclosure;

FIG. 2B is a cross-section of what is illustrated in FIG. 2A;

FIG. 3 illustrates a needle guide system in accordance with illustrative embodiments of the present disclosure; and

FIG. 4 illustrates a method of using a needle guide in accordance with illustrative embodiments of the present disclosure.

Like reference numbers and designations in the various drawings indicate like elements. It should also be noted that the accompanying drawing figures referred to herein are not all drawn to scale, but may be exaggerated to illustrate various aspects of the present disclosure, and in that regard, the drawing figures should not be construed as limiting.

### DETAILED DESCRIPTION

Persons skilled in the art will readily appreciate that various aspects of the present disclosure may be realized by any number of methods and apparatuses configured to perform the intended functions. Stated differently, other methods and apparatuses may be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

Illustrative embodiments of the present disclosure comprise devices, systems and methods for improved dialysis cannulation.

In this regard, while the present disclosure may be described primarily with reference to dialysis cannulation, the present disclosure is not so limited. Indeed, those skilled in the art will appreciate that the principles described herein can be applied to other procedures requiring cannulation including, but not limited to, central venous catheter cannulation.

As will be described in greater detail below, a needle guide in accordance with the present disclosure can provide for a consistent cannulation entry site, entry angle and/or depth. A needle guide in accordance with the present disclosure can also be configured to maintain a drug at the cannulation entry site and/or maintain pressure at the cannulation entry site.

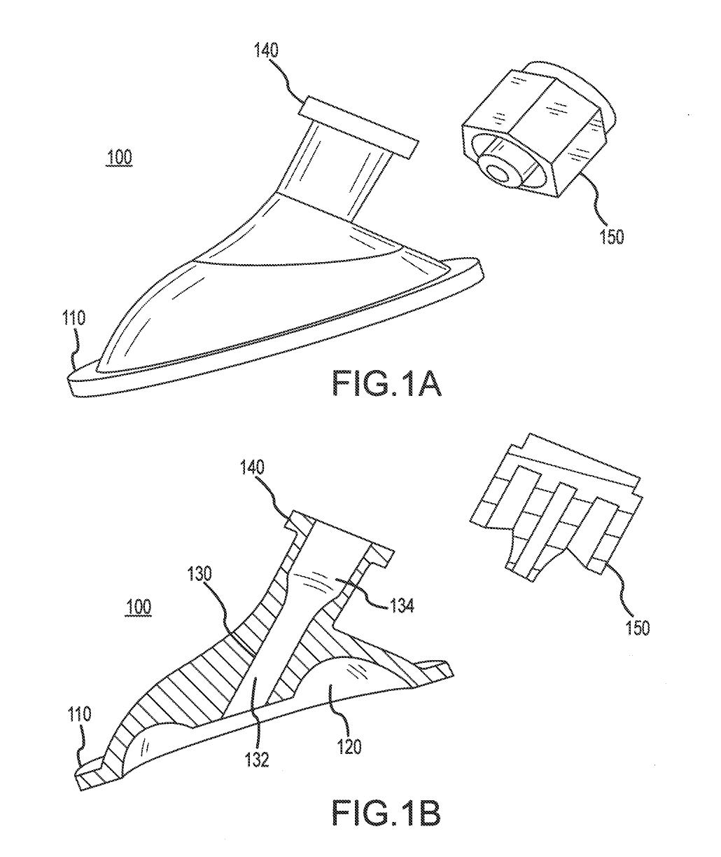

With reference now to FIGS. 1A and 1B, a needle guide 100 in accordance with the present disclosure can comprise a base 110, a cavity 120, a needle path 130, an end portion 140, and a cap 150.

Base 110 is generally configured to provide an interface for needle guide 100 to couple to a patient's skin. In this regard, base 110 can have a generally planar surface, or can have a surface that substantially conforms to, or is flexibly conformable to, a patient's skin. In other embodiments however, base 110 is configured to provide a subdermal interface for needle guide 100 to couple to a patient's vessel, fistula, graft or tissue surface. In such embodiments, base 110 can have a generally planar surface, or can have a surface that substantially conforms to a patient's vessel, fistula, graft or tissue surface.

Base 110 can have any shape including, but not limited to, profiles that are elliptical (e.g., circles, ovals, ellipses, and the like), non-elliptical (e.g., triangles, rectangles, squares, hexagons, trapezoids, pentagons, stars, and the like), or random. Moreover, the cross-section can vary in shape and/or size from end to end.

In accordance with illustrative embodiments, base 110 can be coupled with an adhesive, such as a biocompatible tape, glue or hydrogel. In other embodiments however, base 110 can be coupled with negative pressure (e.g., suction) or suturing. Coupling of base 110 can be temporary or permanent. Coupling of base 110 can be water resistant or waterproof. In general, coupling of base 110 can provide a seal between base 110 and the surface to which base 110 is coupled, wherein the seal prevents movement of base 110 and is closed, or substantially closed, to the transfer of pathogens, liquids and/or pressure.

In accordance with illustrative embodiments, the interior surface of needle guide 100 extends from base 110 to form cavity 120. In illustrative embodiments, cavity 120 partially or completely extends into, or is otherwise located within, base 110. In general, cavity 120 defines a void above the surface to which base 110 is coupled, wherein the void is closed, or substantially closed, to the transfer of pathogens, liquids and/or pressure.

Cavity 120 is empty in some embodiments, while cavity 120 in other embodiments comprises a filler. In general, the filler can be any medium including, but not limited to, a liquid or gel.

Cavity 120 in accordance with the present disclosure can be configured to maintain a drug at the cannulation entry site. In this regard, the filler can be infused with, or otherwise comprise, a therapeutic agent including, but not limited to, a pain reliever, anti-bacterial agent, anticoagulant and/or a combination of the foregoing.

Examples of therapeutic agents comprise antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP)IIbIIIa inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine{cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF) platelet derived growth factor (PDGF), erythropoetin; angiotensin receptor blocker; nitric oxide donors; anti-sense oligionucleotides and combinations thereof; cell cycle inhibitors, mTOR inhibitors, growth factor signal transduction kinase inhibitors, chemical compound, biological molecule, nucleic acids such as DNA and RNA, amino acids, peptide, protein or combinations thereof.

Cavity 120 in accordance with the present disclosure can also be configured to maintain pressure at the cannulation entry site. Thus, the filler can be under pressure within cavity 120, so as to exert pressure at the cannulation entry site when a needle is removed therefrom, and thereby simulate the digital pressure typically applied directly to the cannulation entry site to achieve hemostasis.

As mentioned above, needle guide 100 further comprises needle path 130. In general, needle path 130 defines a bore through which a needle can pass. In this regard, needle bore 132 can have an inner diameter approximately equal to, or slightly larger than, the outer diameter of a needle shaft. Needle path 130 and needle bore 132 may also extend into the tissue. In illustrative embodiments, needle path 130 is circumferentially enclosed between its ends, either partially or completely. In illustrative embodiments, needle path 130 extends into cavity 120, either partially or completely.

In general, needle path 130 also directs a needle through cavity 120 to the cannulation entry site to provide for a consistent cannulation entry site. In this regard, needle path 130 can further be angled to provide for a consistent cannulation entry angle. For example, needle path 130 can be oriented at an angle relative to base 110 of between about 0 and about 90 degrees, or at about 45 degrees. Alternatively, the needle path 130 can be oriented at an angle relative to base 110 of approximately 10 degrees, 20 degrees, 30 degrees, 40 degrees, 50 degrees, 60 degrees, 70 degrees, or 80 degrees or any incremental angle between.

Needle path 130 can further define a flared segment into which a needle hub or adapter can enter, either partially or completely, and stop. In this regard, needle path 130 can provide for a consistent cannulation depth. More particularly, flared segment 134 can comprise a bore (e.g., a cylindrical bore) having a larger diameter than needle bore 132, but having an inner diameter approximately equal to, or slightly larger than, the outer diameter of a needle hub or adapter. In this manner, flared segment 134 can form a needle stop which interacts with a needle hub or adapter inserted into needle path 130 to control the depth of a tip of a needle, to which the needle hub or adapter is attached. Flared segment 134 can be configured to engage with the needle hub or adapter to temporarily or permanently couple them to one another. Such engagement can be threaded, snap fit, tension (interference) fit or the like.

Notwithstanding the foregoing, those skilled in the art will appreciate that flared segment 134 need not be present to control the depth of a tip of a needle. For example, the needle hub or adapter, to which the needle is attached, can interact with any portion of needle path 130 (e.g., needle bore 132) and/or end portion 140, as described below.

In illustrative embodiments of the present disclosure, flared segment 134 serves as a guide to “funnel” or direct a tip of a needle to needle bore 132. In some embodiments, flared segment 134 comprises a material or a coating that does not dull a tip of a needle when directed to needle bore 132. Indeed, flared segment 134 can comprise a material or a coating that sharpens a tip of a needle when directed to needle bore 132.

In illustrative embodiments of the present disclosure, needle path 130 comprises a valve. The valve can be a self-sealing, crumple valve that opens and closes upon inserting and removing a needle there through. In other illustrative embodiments, the valve can be manually opened and closed. The valve can be positioned at an end of, within, or along the needle path. The addition of a check-valve component ensures that excessive bleeding will be arrested in the case of inadvertent or premature needle removal. This feature could be extremely important in the case of nocturnal home hemodialysis, where the needle may inadvertently be pulled out when the patient moves body position while sleeping.

Needle guide 100 further comprises end portion 140, which is generally at an end of needle path 130. In some embodiments, end portion 140 can be configured to engage with cap 150 to temporarily or permanently couple them to one another. Such engagement can be threaded, snap fit, tension fit or the like.

Optionally, needle guide 100 further comprises cap 150 for when needle guide 100 is not in use. As mentioned above, cap 150 can be configured to engage with end portion 140 to temporarily or permanently couple them to one another. Cap 150 may also be configured with a check-valve for home hemodialysis Such engagement can be threaded, snap fit, tension fit or the like.

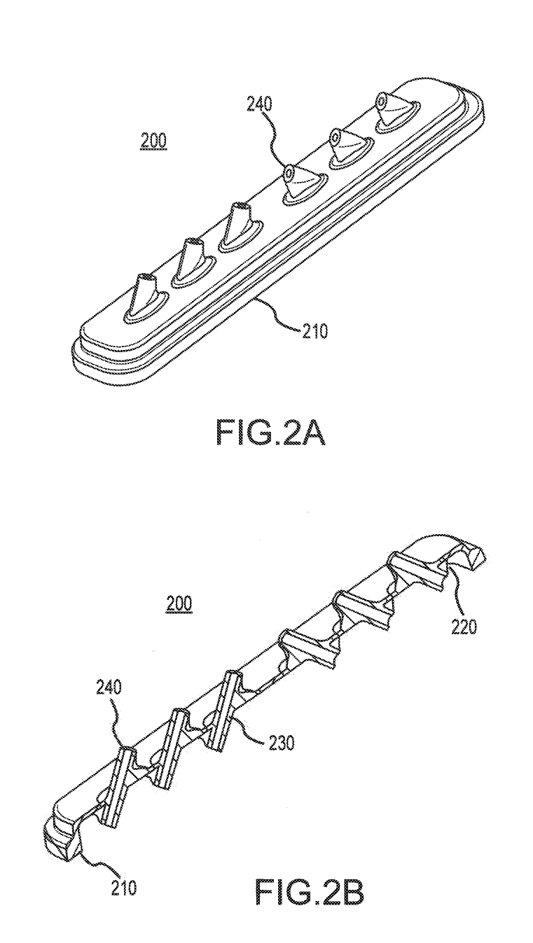

With reference now to FIGS. 2A and 2B, needle guide 200 can comprise a plurality of needle paths 230, end portions 240, and caps (not shown), each of which shares a common base 210 and cavity 220. In this regard, needle guide 200 can facilitate both blood flowing to a dialysis machine, and blood returning to a patient from the dialysis machine. In this same manner, needle guide 200 can also facilitate multiple cannulation entry sites, entry angles and/or depths.

In accordance with illustrative embodiments, a needle guide as described herein is comprised of a thermoplastic or thermoset material, for example, molded or extruded. Other materials may be used including, but not limited to, rigid materials, such as plastics, metals, alloys and the like, or flexible materials, such as silicones, nitriles, nylons, polycarbonates, polyethylenes, polypropylenes and the like. In some embodiments, a material having natural anti-bacterial properties can be used, for example, copper or silver.

Each of the base, cavity, needle path(s), and end portion(s) can be temporarily or permanently coupled to one or more of the others by adhesion, compression fit, threading, suture, glue, thermal bonding, nitinol or other shape memory clips, and the like. Likewise, any plurality of the base, cavity, needle path(s), and end portion(s) can be integral one with another. For example, in example embodiments, a membrane and a lumen comprise a single piece formed from a single mold (e.g., injection mold), extruded together, etc.

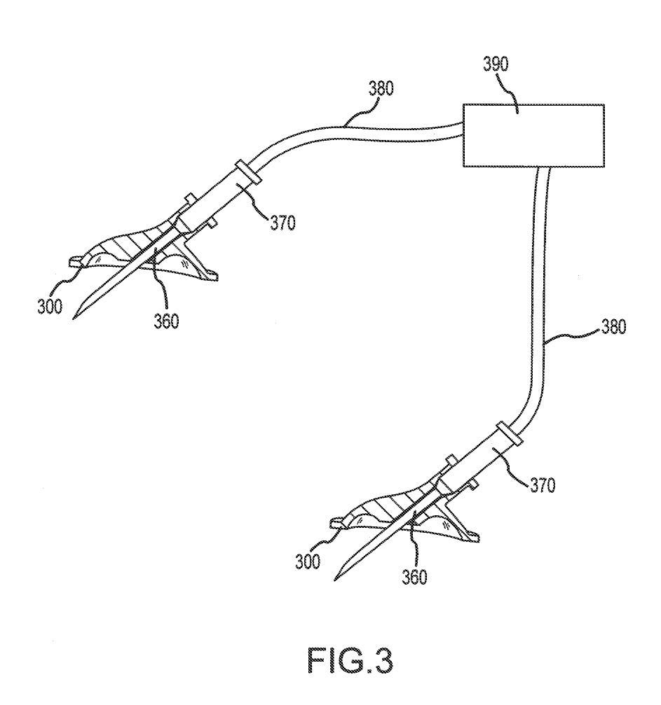

With reference to FIG. 3, a needle guide system in accordance with the present disclosure can comprise one or more needle guides 300 as described above, one or more needles 360, one or more luer lock fittings 370 (luer may be internally threaded, externally threaded, or some combination thereof), tubing 380, and a dialysis machine 390.

The term “needle,” as used herein, is generally any longitudinally extending structure with a lumen therethrough. Thus, elongate elements include but are not limited to introducer sheaths, introducers, sheaths, tubes with lumens (e.g., catheters), hollow wires (e.g., guidewires), hollow stylets, metal tubes (e.g., hypotubes), and polymer tubes. Needles can be any material and can have any cross-sectional shape including, but not limited to, profiles that are elliptical (e.g., circles, ovals, ellipses, and the like), non-elliptical (e.g., triangles, rectangles, squares, hexagons, trapezoids, pentagons, stars, and the like), or random. Moreover, the cross-section can vary in shape and/or size from end to end.

In this regard, and as mentioned above, embodiments of the present disclosure can be applied to other procedures requiring cannulation including, but not limited to, central venous catheter cannulation. In such an embodiment, a central venous catheter can enter a patient's skin within a cavity of a needle guide as described above. The cavity can be closed, or substantially closed, to the transfer of pathogens, liquids and/or pressure. In addition, the cavity can be configured to maintain a drug at the catheter entry site. In this manner, the incidence of infection and the skin-interface site can be minimized.

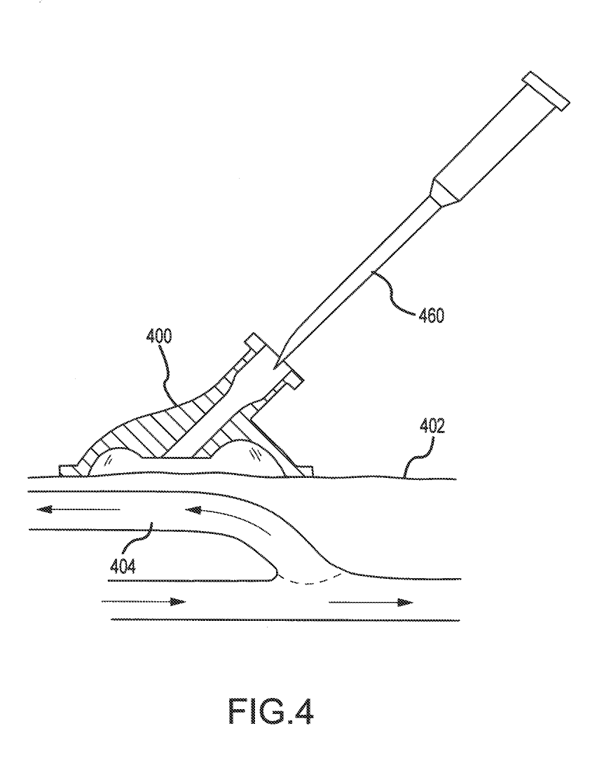

With reference now to FIG. 4, a method of using a needle guide in accordance with the present disclosure can comprise providing a needle guide 400 as described above, aligning needle guide 400 with a desired vessel, fistula, or graft 404 of a patient, coupling needle guide 400 to the patient's skin 402, and inserting a needle 460 through needle guide 400 and into desired vessel, fistula, or graft 404.

The foregoing steps can be repeated as necessary, for example, to facilitate both blood flowing to a dialysis machine, and blood returning to a patient from the dialysis machine.

Once a dialysis procedure has been completed, needle 460 can be removed through needle guide 400 from desired vessel, fistula, or graft 404. Finally, once no additional dialysis procedures are needed or a different cannulation entry site is to be selected, needle guide 400 can be removed from the patient's skin 402.

While this specification contains many specific implementation details, these should not be construed as limitations on the scope of any devices, methods, and systems discussed herein, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims.

What is claimed is:

1. A needle guide comprising:
a base; and
a needle path coupled to the base and comprising a needle bore, a flared segment, and a needle stop,
wherein the needle stop interacts with a hub of a needle inserted into the needle path to control penetration depth of a tip of the needle,
wherein the flared segment comprises a cylindrical bore having a larger diameter than the needle bore, and
wherein the needle path is oriented at an angle relative to the base of between about 0 and about 90 degrees.

2. The needle guide of claim 1, wherein the needle path is circumferentially enclosed between its ends.

3. The needle guide of claim 1, wherein the needle path is oriented at an angle of about 45 degrees.

4. The needle guide of claim 1, wherein the needle path is oriented at an angle of about 50 degrees.

5. The needle guide of claim 1, wherein the flared segment forms the needle stop.

6. The needle guide of claim 1, further comprising a cavity located within the base.

7. The needle guide of claim 6, further comprising a hydrogel within the cavity of the base.

8. The needle guide of claim 7, wherein the hydrogel is infused with a therapeutic agent.

9. The needle guide of claim 7, wherein the hydrogel is pressurized within the cavity of the base.

10. The needle guide of claim 1, wherein the needle guide comprises a thermoplastic.

11. The needle guide of claim 1, wherein the base is a circular or oval shape.

12. The needle guide of claim 1, wherein the base is attached to a skin surface of a patient through one of a tape, adhesive, hydrogel, and suction.

13. The needle guide of claim 1, wherein the base is implanted under a skin surface and attached to a vessel, fistula, graft or tissue surface.

14. The needle guide of claim 1, further comprising an end portion located at an end of the flared segment and configured to receive a cap.

15. The needle guide of claim 14, wherein the end portion and the cap are threaded in complimentary thread patterns to each other.

16. The needle guide of claim 1, further comprising a self-sealing valve positioned at an end of the needle path.

17. The needle guide of claim 1 further comprising a self-sealing valve positioned within the needle path.

18. The needle guide of claim 1 comprising a plurality of needle paths.

19. A system for dialysis comprising:
a plurality of needle guides wherein each needle guide comprises:
a base; and
a needle path coupled to the base and comprising a needle bore, a flared segment, and a needle stop,
wherein the needle stop interacts with a hub of a needle inserted into the needle path to control penetration depth of a tip of the needle,
wherein the flared segment comprises a cylindrical bore having a larger diameter than the needle bore,
wherein the needle path is oriented at an angle relative to the base of between about 0 and about 90 degrees; and
wherein said system further comprises a plurality of needles and luer lock fittings, tubing and a dialysis machine.

20. A method for inserting a needle into a vascular system of a patient comprising:
providing a needle guide comprising a base and a needle path coupled to the base and comprising a needle bore, a flared segment, and a needle stop, wherein the needle stop interacts with a hub of a needle inserted into the needle path to control penetration depth of a tip of the needle, wherein the flared segment comprises a cylindrical bore having a larger diameter than the needle bore, and wherein the needle path is oriented at an angle relative to the base of between about 0 and about 90 degrees;
aligning the needle guide with a desired vessel of a patient; and
inserting a needle through the needle guide and into the desired vessel of the patient.

21. The method of claim 20, wherein the needle path is circumferentially enclosed between its ends.

22. The method of claim 20, wherein the flared segment forms the needle stop.

23. The method of claim 20, further comprising the step of securing the needle guide to a skin surface of the patient.

24. The method of claim 20, further comprising the steps of withdrawing the needle from the needle guide and coupling a cap to the needle guide.

25. The method of claim 20, wherein the needle guide comprises a cavity located within the base and a hydrogel within the cavity of the base.

26. The method of claim 25, wherein the hydrogel is infused with a therapeutic agent.

27. The method of claim 25, wherein the hydrogel is pressurized within the cavity.

28. The method of claim 20, wherein the needle guide comprises a thermoplastic.

29. The method of claim 20, wherein the needle guide comprises an end portion located at an end of the flared segment and configured to receive a cap.

30. The method of claim 29, wherein the end portion and the cap of the needle guide are threaded in complimentary thread patterns to each other.

31. The method of claim 20, wherein the needle guide comprises a self-sealing valve positioned along the needle path.

32. The method of claim 20 comprising a plurality of needle paths.
